(19) United States
(12) Patent Application Publication
(10) Pub. No.: US 2025/0391570 A1
(43) Pub. Date: Dec. 25, 2025

(54) Mental Health Measurement And Guidance System Based On Wearable Device Data

(71) Applicant: Zepp, Inc., Cupertino, CA (US)

(72) Inventors: Mohammad M. Herzallah, Cupertino, CA (US); Mengfan Tang, Cupertino, CA (US); Kongqiao Wang, Cupertino, CA (US)

(21) Appl. No.: 18/783,750

(22) Filed: Jul. 25, 2024

Related U.S. Application Data
(60) Provisional application No. 63/662,194, filed on Jun. 20, 2024.

(51) Int. Cl. G16H 50/30

(52) U.S. Cl. CPC G16H 50/30

(57) ABSTRACT

A method of dynamically monitoring emotions of a user using a wearable device. The method includes detecting one or more physiological signals associated with a user and determining, in a first layer of the method, one or more detected emotions associated with the one or more physiological signals detected. The method also includes determining, in a second layer of the method, one or more symptoms of the user, whereby the one or more symptoms are based on the one or more detected emotions. The method further includes determining, in a third layer of the method, one or more mental wellbeing metrics associated with a mental wellbeing of the user, whereby the one or more mental wellbeing metrics are based on the one or more detected emotions and the one or more symptoms.

### CROSS-REFERENCE TO RELATED APPLICATIONS

This application claims priority to U.S. Provisional Application Ser. No. 63/662,194, filed on Jun. 20, 2024, the contents of which are incorporated herein by reference in their entirety.

### TECHNICAL FIELD

This application relates to wearable computing, and in particular, to dynamic tracking and guidance of mental health parameters based upon wearable computing input.

### BACKGROUND

Modern technologies have provided users with wearable computing devices configured to sense and track a user's physical parameters. Based upon such physical parameters of the user, the wearable computing devices of associated computer-based system may compute health-related analyses to track the health of the user.

Current wearable computing devices may also compute analyses to correlate the detected physical parameters of the user with emotions of the user. For example, the wearable computing devices may detect a change in one or more physical parameters of the user, and the wearable computing devices may then associate such changes in the one or more physical parameters with one or more possible emotions of the user.

### SUMMARY

Disclosed herein are implementations of methods, apparatuses, and systems for mental health measurement and guidance.

In one aspect of the disclosure, a method of dynamically monitoring emotions of a user using a wearable device is disclosed. The method includes detecting, by the wearable device when worn by the user, one or more physiological signals associated with a user, and determining, by a processor in a first layer of the method, one or more detected emotions associated with the one or more health physiological signals detected. Responsive to determining the one or more detected emotions, the method includes determining, by the processor in a second layer of the method, one or more symptoms of the user, whereby the one or more symptoms are based on the one or more detected emotions. Additionally, responsive to determining the one or more symptoms, the method also includes determining, by the processor in a third layer of the method, one or more mental wellbeing metrics associated with a mental wellbeing of the user, whereby the one or more mental wellbeing metrics are based on the one or more detected emotions and the one or more symptoms.

In certain configurations, responsive to determining the one or more detected emotions, the method may further include prompting the user to confirm or reject the one or more detected emotions. The one or more detected emotions may be cumulatively tracked to create a log when the user confirms the one or more detected emotions. Responsive to the user rejecting the one or more detected emotions, the method may also include modifying one or more parameters used by the processor to determine the one or more detected emotions. If the user rejects the one or more detected emotions, the user may be prompted to input one or more input emotions, whereby the one or more input emotions may be different than the one or more detected emotions.

In certain configurations, responsive to determining the one or more symptoms, the method may further include prompting the user to confirm or reject the one or more symptoms. Additionally, responsive to determining the one or more mental wellbeing metrics, the method may further include prompting the user to confirm or reject the one or more wellbeing metrics.

In certain configurations, the method may further include providing a mental health plan to the user based upon at least one of the one or more detected emotions, the one or more symptoms, and the one or more mental wellbeing metrics. The mental health plan may include at least one of recommendations, activities, actions, and mental health information.

In certain configurations, the method may further include cumulatively tracking by the processor in the first layer, the one or more detected emotions within a first time interval to obtain a first emotion summary and within a second time interval to obtain a second emotion summary. The one or more symptoms may be determined based on the first emotion summary and the second emotion summary such that the one or more symptoms are based upon a third time interval that combines the first time interval and the second time interval.

In certain configurations, prior to detecting the one or more physiological signals associated with the user, the method may further include receiving, from the user as an input, an emotional baseline that includes one or more expected emotions or a response to a wellbeing question to assess a validity of the one or more detected emotions.

In certain configurations, the one or more detected emotions may be categorized based upon an associated arousal signal and an associated valence signal that are derived from the one or more physiological signals. A combination of the associated arousal signal and the associated valence signal may be unique for each of the one or more detected emotions.

In certain configurations, the method may further include determining, by the processor, at least one of an intensity of the one or more detected emotions and an intensity of the one or more symptoms.

In another aspect of the disclosure, a wearable device for dynamically monitoring emotions of a user wearing the wearable device is disclosed. The wearable device includes a non-transitory memory and a processor configured to execute instructions stored in the non-transitory memory. The processor is configured to execute instructions stored in the non-transitory memory to detect, by the wearable device, one or more physiological signals associated with a user, and determine one or more detected emotions associated with the one or more physiological signals detected. Responsive to determining the one or more detected emotions, the processor is configured to execute instructions stored in the non-transitory memory to determine one or more symptoms of the user, whereby the one or more symptoms are based on the one or more detected emotions. Responsive to determining the one or more symptoms, the process is configured to execute instructions stored in the non-transitory memory to determine one or more mental wellbeing metrics associated with a mental wellbeing of the user, whereby the one or more mental wellbeing metrics are based on the one or more detected emotions and the one or more symptoms.

In certain configurations, responsive to determining the one or more detected emotions, the processor may be further configured to execute instructions stored in the non-transitory memory to prompt the user to confirm or reject the one or more detected emotions. The one or more detected emotions may be cumulatively tracked to create a log when the user confirms the one or more detected emotions. Responsive to the user rejecting the one or more detected emotions, the processor may be further configured to execute the instructions stored in the non-transitory memory to modify one or more parameters used by the processor to determine the one or more detected emotions.

In certain configurations, the log may be created to cumulatively track the one or more detected emotions on a momentary basis, an event-related basis, a daily basis, a weekly basis, and a monthly basis. Moreover, the processor may be further configured to execute instructions stored in the non-transitory memory to provide a mental health plan to the user based upon at least one of the one or more detected emotions, the one or more symptoms, and the one or more mental wellbeing metrics. The mental health plan may include at least one of recommendations, activities, actions, and mental health information. The one or more detected emotions may include one or more emotion categories represented by fear, sadness, happiness, and excitement.

In certain configurations, prior to detecting the one or more physiological signals associated with the user, the processor may be further configured to execute the instructions stored in the non-transitory memory to receive, from the user as an input, an emotional baseline that includes one or more expected emotions or a response to a wellbeing question to assess a validity of the one or more detected emotions.

In another aspect of the disclosure, a non-transitory computer-readable storage medium configured to store computer programs for dynamically monitoring emotions of a user using a wearable device is disclosed. The computer programs include instructions executable by a processor to detect, by the wearable device when worn by the user, one or more physiological signals associated with a user, and determine one or more detected emotions associated with the one or more physiological signals detected. The one or more detected emotions are cumulatively tracked to create a log. Responsive to determining the one or more detected emotions, the computer programs may also include instructions executable by the processor to determine one or more symptoms of the user, whereby the one or more symptoms are based on the one or more detected emotions. Responsive to determining the one or more symptoms, the computer programs may also include instructions executable by the processor to determine one or more mental wellbeing metrics associated with a mental wellbeing of the user, whereby the one or more mental wellbeing metrics are based on the one or more detected emotions and the one or more symptoms.

In certain configurations, the computer programs may further include instructions executable by the processor to provide a mental health plan to the user based on at least one of the one or more detected emotions, the one or more symptoms, and the one or more mental wellbeing metrics. The mental health plan may include at least one of recommendations, activities, actions, and mental health information.

In certain configurations, prior to detecting the one or more physiological signals associated with the user, the computer programs may further include instructions executable by the processor to receive, from the user as an input, an emotional baseline that includes one or more expected emotions or a response to a wellbeing question to assess a validity of the one or more detected emotions.

In certain configurations, the one or more detected emotions may be categorized based upon an associated arousal signal and an associated valence signal that are derived from the one or more physiological signals. A combination of the associated arousal signal and the associated valence signal may be unique for each of the one or more detected emotions.

### BRIEF DESCRIPTION OF DRAWINGS

FIG. 1 is a perspective view of an example of a wearable device in accordance with the present teachings.

FIG. 2 is a block diagram of an example of a computing device that may be used with or incorporated into a wearable device in accordance with the present teachings.

FIG. 3 is an example of a mental health assessment chart in accordance with the present teachings.

FIG. 4 is a flow diagram illustrating dynamic tracking of emotions of an individual.

FIG. 5 is a flow diagram of an example of cross-validating emotions with an individual and providing a mental health plan to the individual.

FIG. 6 is an example of a user interface for dynamic tracking of emotions of an individual.

FIG. 7 is a flow diagram of an example of a method for dynamically monitoring emotions of a user using a wearable device.

### DETAILED DESCRIPTION

Many portable devices and systems have been developed to monitor physiological conditions of an individual. One area of interest in the use of physiological monitors is personal wellness and mental health. Mental health is an integral part of the overall health of an individual and may be evaluated to determine a mental health state of the individual at a given point in time. While assessment of mental health states may be conventionally completed by conducting interviews and questionnaires (e.g., by a mental health expert, such as a medical professional), some portable devices may be utilized in an attempt to associate physiological conditions of the individual (e.g., physiological conditions of the individual detected by the portable devices) with a mental health state of the individual. For example, a portable device may be worn by the individual and may detect one or more physiological conditions of the individual (e.g., heart rate, heart rate variability (HRV), skin conductance, respiration rate, blood pressure, pupil dilation, body temperature, etc.), and the one or more detected physiological conditions may be evaluated to determine an emotion of the individual. However, such portable devices and systems thereof may have certain shortcomings that hinder the overall usability of the portable devices.

By way of example, conventional portable devices (e.g., conventional wearable devices) may be sufficient to report certain metrics associated with the individual, such as general emotions associated with one or more physiological conditions detected by the conventional portable devices, the metrics reported may often be incomplete, inaccurate, or too broadly spanning to provide the sufficient data needed to complete a comprehensive evaluation of the individual's mental health state. Additionally, the conventional portable devices may also automatically associate certain physiological conditions detected with certain emotions based upon a default evaluation protocol (e.g., a pre-defined algorithm, process, software, etc.). Thus, the emotions determined based on the physiological conditions detected may often be inaccurate and may be unable to reflect the actual physiological and emotional feelings subjectively sensed by the individual.

The present teachings help to solve the aforementioned difficulties. In particular, the present teachings provide an evaluation system, which may be used by or in conjunction with one or more portable devices (e.g., wearable devices), that dynamically monitors and tracks a mental health state of an individual. The present teachings may determine one or more emotions based upon one or more physiological symptoms detected by the portable device, whereby the one or more determined emotions may be cross-validated by the individual to confirm whether the one or more determined emotions accurately reflect the physiological and emotional feelings subjectively sensed by the individual. Based on such cross-validation, the evaluation system may more accurately track the emotions of the individual and may modify or adjust the determined emotions based upon manual input by the individual. The evaluation system may further track (e.g., log) the emotions of the individual and provide information to the individual based upon the tracked emotions. For example, the evaluation system may track the emotions of the individual on a momentary and/or an event-related basis, a daily basis, a weekly basis, and a monthly basis, and the evaluation system may provide (e.g., via the portable device) a mental health plan to the individual based upon the emotions of the individual. The mental health plan may provide recommended activities and/or actions to the individual, may provide mental health data and/or information to the individual, or a combination thereof.

Based on the above, the present disclosure provides a data-drive approach that utilizes data from a wearable device (e.g., a smart watch worn by the individual) to detect and reveal mental health patterns of the individual that could positively and/or negatively affect an individual's overall mental wellbeing. Implementations of this disclosure aim to help the individual successfully maintain a healthy mental state. For example, based on the emotions tracked by the evaluation system, the evaluation system may provide a mental health plan to the individual to improve any emotions that may negatively impact the individual, thereby improving the overall mental state of the individual. That is, the evaluation system in accordance with the present teachings may be an evaluation and guidance system for the mental health of the individual.

By dynamically monitoring physiological conditions of the individual and receiving feedback directly from the individual (e.g., cross-validation of emotions determined by the evaluation system), the overall accuracy of the emotions tracked may be improved and the resultant mental health plan provided to the individual may be dynamically adjusted on a frequent and/or regular basis as needed to account for any discrepancies between the emotions determined by the evaluation system and the emotions subjectively sensed by the individual. Thus, the mental health plan provided to the individual may be tailored specifically to the individual and may produce a holistic view of the mental health of the individual that accounts for both objective data (e.g., the emotions determined by the evaluation system based upon the physiological conditions detected by the portable device) and subjective data (e.g., feedback from the individual).

Moreover, according to implementations of this disclosure, sensor data for the individual can be collected, for example, by the portable device (e.g., the wearable device). The collected sensor data for the individual may be extracted to obtain physiological conditions of the individual, such as heart rate and/or heart rate variability (HRV), sleep duration, sleep interruptions, body temperature, skin conductance, respiration rate, blood pressure, pupil dilation, an electrocardiogram (ECG), an electroencephalogram (EEG), an electrooculogram (EOG), an electromyogram (EMG), or a combination thereof. Based upon the data extracted and physiological conditions determined, mental health conditions of the individual (e.g., emotions felt by the individual, an overall mental health state of the individual, etc.) may be accurately determined and can be used to provide a mental health plan to the individual, which may be followed by the individual to reach an overall mental health goal, such as a healthier overall mental wellbeing.

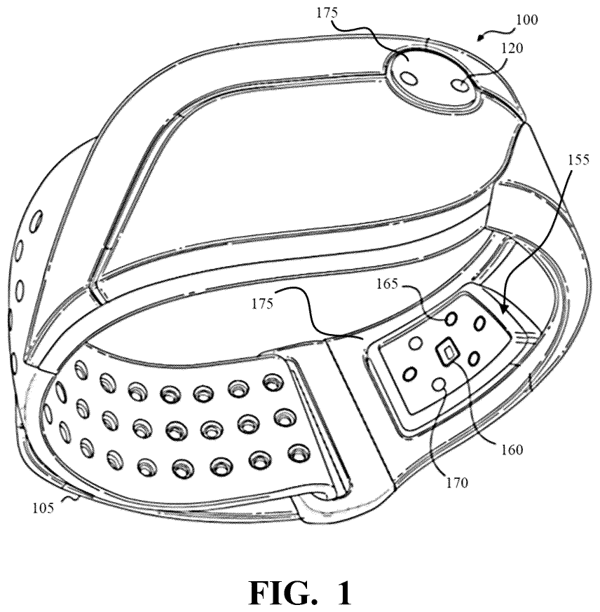

FIG. 1 depicts a perspective view of an example device 100 according to some implementations of this disclosure. The device 100 may be a wearable device worn by an individual (also referred to herein as a user) to at least one of sense, collect, monitor, analyze, or display information pertaining to one or more of a physiological parameter of the individual or an environmental parameter captured by the device 100 in a vicinity of the individual. The device 100 can include, for example, a head mounted device, a wristband, a ring, a strap (e.g., a chest strap), headphones or a wristwatch. Although depicted in FIG. 1 as a wristwatch, the device 100 can include the wearable device configured for positioning at a user's wrist, arm, finger, chest, another extremity of the user, or some other area of the user's body, such as a wearable camera. For example, the device 100 can be a wearable camera having an image sensor with capabilities such as high-speed shooting performance, low-light or dark shooting performance, high image quality, or anti-shake performance, among other things. In addition, the wearable camera can be equipped with other sensors as discussed below (e.g., a photoplethysmography (PPG) sensor to detect heart rate, altitude sensor, a temperature sensor, a humidity sensor, etc.).

The device 100 may include sensors and processing tools for detecting, collecting, processing, or displaying one or more physiological parameters of the individual and/or other information that may or may not be related to health, wellness (e.g., mental health and wellness), exercise, sleep, or physical training sessions (e.g., characteristic information, education information, etc.). The physiological parameter can include at least one of: heart rate, heart rate variability (HRV), blood pressure, blood glucose level, respiration rate, body temperature, electrocardiogram (ECG) measurements, electroencephalogram (EEG) measurements, electrooculogram (EOG) readings, electromyogram (EMG) readings, sleep state, sleep phase, mental state, stress state, or other physiological information that can be measured for the individual.

The device 100 may also include sensors and processing tools for detecting, collecting, processing, or displaying one or more environmental parameters captured by the device 100 in a vicinity of the individual.

The environmental parameter can include, for example, positioning information, location, altitude, temperature, humidity, environmental light, weather, environmental pollution index such as PM2.5 particulate matter content or CO2/CO content, which can be captured by, for example, one or more environmental sensors of the device 100. The environmental parameter can also include motion data such as motion tracks from a GPS sensor and/or a motion sensor (e.g., one or more of an accelerometer, gyroscope, magnetometer, etc.) or a barometer to record additional measurement data such as altitude. The environmental parameter can include at least one of: altitude, GPS location, ambient temperature, ambient humidity, ambient light, ambient noise index, an environmental pollution index, or other environmental parameter in the vicinity of the individual that can be captured by the at least one wearable device.

The device 100 may further include one or more communication modules. One or more communication modules may also communicate with other devices such as a personal device of the user (such as a handheld device, a smart phone, a tablet, a laptop computer, a desktop computer, or the like) or a server (such as a cloud-based server). The communications can be transmitted wirelessly (e.g., via Bluetooth, RF signal, Wi-Fi signal, near field communications, etc.) or through one or more electrical connections embedded in the band 105. Any analog information collected or analyzed can be translated to digital information for reducing the size of information transfers between modules.

As shown in FIG. 1, the device 100 can include a sensor unit 155 including at least one of, but not limited to, an image sensor, such as a camera (not shown), one or more physiological sensors such as a PPG, EEG, EOG, or EMG sensor including one or more optical detectors 160 and one or more light sources 165, one or more contact pressure/tonometry sensors 170, or one or more motion sensors including at least one of the one or more gyroscopes or accelerometers 175. These sensors are only illustrative of the possibilities, however, and additional or alternative sensors such as one or more acoustic sensors, electromagnetic sensors, ECG electrodes 120, bio impedance sensors, or galvanic skin response, or a combination thereof may be included. Though not depicted in the view shown in FIG. 1, the device 100 may also include one or more such sensors and components on its inside surface (i.e., the surface in contact with the user's tissue or targeted area). It should be understood that the device 100 can be implemented with a different configuration of the sensor unit 155 from what is depicted in FIG. 1 or the examples of the disclosure.

The location of the sensor unit 155 or the location of one or more sensor components of the sensor unit 155 with respect to the user's tissue may be customized to account for differences in body type across a group of users or placement in different locations on a user.

The displacement values and additional data collected from the sensor unit 155 may assist a non-transitory computer readable medium or processor in isolating various physiological conditions (e.g., heart beats, respiration, etc.). The processor may receive data from the sensor unit 155. The processor may dynamically filter the data. The processor may analyze the data without regard to a position of the device relative to the user or a position of the user. The processor may filter unwanted signals and isolate only desired signals. For example, the processor may learn which signals are of interest (e.g., which signals are associated with physiological conditions of interest, which may form a basis to determine one or more emotions of a user) and the process may analyze only those signals of interest. The processor may be in communication with or include a non-transitory computer-readable medium.

The sensor unit 155 can be configured to continuously collect data from a user. However, certain techniques can be employed to reduce power consumption and conserve battery life of the device 100. For example, while the PPG sensor can be used to continuously monitor blood flow of the user, the ECG electrodes 120 can be used periodically or intermittently to collect potentially more accurate blood flow information which can be used to supplement or calibrate the PPG measurements collected and analyzed by the processor.

For example, when the data from one or more accelerometers or gyroscopic components of the device 100 indicates that a user is still or at rest, one or more sensors of the device 100, such as the PPG sensor, which consumes more power than the one or more accelerometers or gyroscopic components, may be turned off to conserve power consumption. However, when the data from the one or more accelerometers or gyroscopic components of the device 100 indicates that the user is exercising, the one or more sensors of the device 100, such as the PPG sensor, may be turned on to measure the heart rate and/or other physiological parameters of the user. In another example, when the data from one or more accelerometers or gyroscopic components of the device 100 indicates that a user is sleeping and the sleep analysis function is turned on, the one or more sensors, such as the PPG sensor, may still need to be turned on even though the movement of the user from the one or more accelerometers or gyroscopic components of the device 100 is minimal during sleep.

The device 100 may also include an input and/or an output unit, such as a display unit (not shown), sound unit, tactile unit or the like, for communicating information to the user (i.e., the wearer of the device 100). The display unit may be configured to display the images or videos captured by the sensors such as the image sensor, notifications or alerts. The display unit may be an LED indicator including a plurality of LEDs, each a different color. The LED indicator can be configured to illuminate in different colors depending on the information being conveyed. For example, where the device 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees the device 100 through the user's peripheral vision (e.g., while exercising).

The display unit may include a display screen for displaying images, characters, graphs, waveforms, or a combination thereof to the user or a medical professional. By way of example, the device 100 may be configured to detect one or more physiological conditions of the user and provide a mental health assessment of the user (e.g., determine an emotion of the user), whereby the mental health assessment may be or may include information displayed on the display screen. The display unit may further include one or more hard or soft buttons or switches configured to accept input by the user. Similarly, the display screen may be a touch screen configured to accept input by the user. The display unit may also switch or be toggled between displaying information.

The physiological or environmental information discussed above may be graphically displayed or represented on a display (not shown) of the device 100. The graphical display may be provided as an output. The output may include physiological or environmental information of a user. For example, the information collected may be categorized and then graphically represented as one or more outputs. The output may include alert, guidance or suggestion to the user. The output may also include education information pertaining to topics of interest for the user.

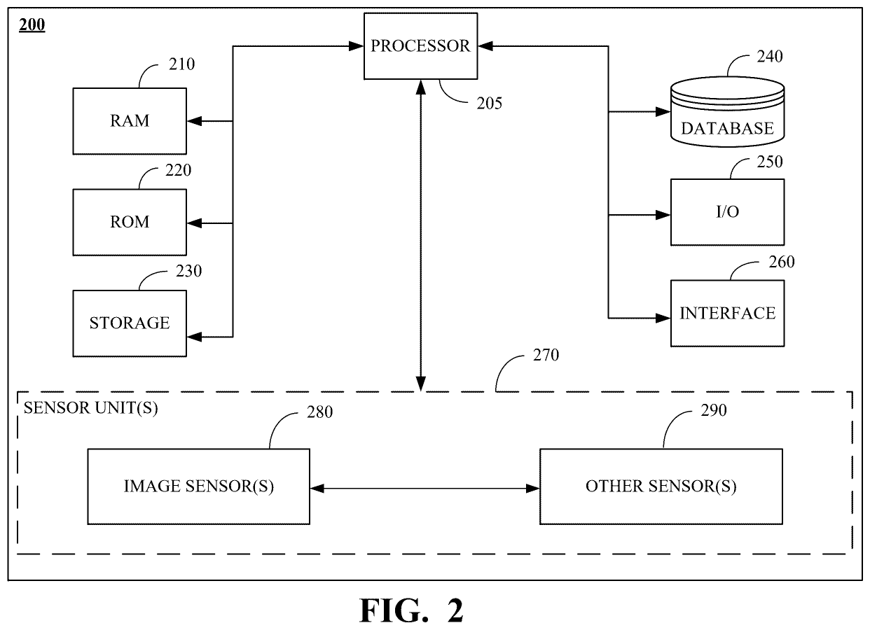

FIG. 2 depicts an example of a computing device 200 that may be used with or incorporated into a wearable device. The computing device 200 is representative of the type of computing device that may be present in or used in conjunction with at least some aspects of the device 100, or any other device comprising electronic circuitry. For example, the computing device 200 may be used in conjunction with any one or more of transmitting signals to and from the one or more optical sensors or acoustical sensors, sensing or detecting signals received by one or more sensors of the device 100, processing received signals from one or more components or modules of the device 100 or a secondary device, and storing, transmitting, or displaying information. The computing device 200 may be or may be included within the device 100. The computing device 200 may be a mobile terminal or remote device that is in communication with the device 100. The computing device 200, the device 100, or both may be in communication with a server (e.g., a cloud-based server). For example, the computing device 200 may be a separate device (e.g., a mobile terminal device) from the device 100, and both the computing device 200 and the device 100 may be in direct communication with the server. Alternatively, the computing device 200 may be in direct communication with the server and the device 100 may be in communication with the server via the computing device 200. It should also be noted that the computing device 200 is illustrative only and does not exclude the possibility of another process- or controller-based system being used in or with any of the aforementioned aspects of the device 100.

In one aspect, the computing device 200 may include one or more hardware and/or software components configured to execute software programs, such as software for obtaining, storing, processing, and analyzing signals, data, or both. For example, the computing device 200 may include one or more hardware components such as, for example, a processor 205, a random-access memory (RAM) 210, a read-only memory (ROM) 220, a storage 230, a database 240, one or more input/output (I/O) modules 250, an interface 260, and one or more sensors 270.

Alternatively, and/or additionally, the computing device 200 may include one or more software components such as, for example, a computer-readable medium including computer-executable instructions for performing techniques or implement functions of tools consistent with certain disclosed embodiments. It is contemplated that one or more of the hardware components listed above may be implemented using software. For example, the storage 230 may include a software partition associated with one or more other hardware components of the computing device 200. The computing device 200 may include additional, fewer, and/or different components than those listed above. It is understood that the components listed above are illustrative only and not intended to be limiting or exclude suitable alternatives or additional components.

The processor 205 may include one or more processors, each configured to execute instructions and process data to perform one or more functions associated with the computing device 200. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 2, the processor 205 may be communicatively coupled to the RAM 210, the ROM 220, the storage 230, the database 240, the I/O module 250, the interface 260, and the one or more sensors 270. The processor 205 may be configured to execute sequences of computer program instructions to perform various processes, which will be described in detail below. The computer program instructions may be loaded into the RAM 210 for execution by the processor 205.

The RAM 210 and the ROM 220 may each include one or more devices for storing information associated with an operation of the computing device 200 and/or the processor 205. For example, the ROM 220, may include a memory device configured to access and store information associated with the computing device 200, including information for identifying, initializing, and monitoring the operation of one or more components and subsystems of the computing device 200. The RAM 210 may include a memory device for storing data associated with one or more operations of the processor 205. For example, the ROM 220 may load instructions into the RAM 210 for execution by the processor 205.

The storage 230 may include any type of storage device configured to store information that the processor 205 may use to perform processes consistent with the disclosed embodiments.

The database 240 may include one or more software and/or hardware components that cooperate to store, organize, filter, and/or arrange data used by the computing device 200 and/or the processor 205. For example, the database 240 may include user profile information, historical activity and user-specific information (e.g., historical information pertaining to the mental health of a specific user), physiological parameter information, predetermined menu/display options, and other user preferences. Alternatively, the database 240 may store additional and/or different information. For example, the database 240 may include information to establish a machine learning model such as a large language model (LLM) that can receive inputs from the I/O module 250 or sensor(s) 270.

The I/O module 250 may include one or more components configured to communicate information with a user associated with the computing device 200. For example, the I/O module 250 may include one or more buttons, switches, or touchscreens to allow a user to input parameters associated with the computing device 200. The I/O module 250 may also include a display including a graphical user interface (GUI) and/or one or more light sources for outputting information to the user. The I/O module 250 may also include one or more communication channels for connecting the computing device 200 to one or more secondary or peripheral devices such as, for example, a desktop computer, a laptop, a tablet, a smart phone, a flash drive, or a printer, to allow a user to input data to or output data from the computing device 200.

The interface 260 may include one or more components configured to transmit and receive data via a communication network, such as the internet, a local area network, a workstation peer-to-peer network, a direct link network, a wireless network, or any other suitable communication channel. For example, the interface 260 may include one or more modulators, demodulators, multiplexers, demultiplexers, network communication devices, wireless devices, antennas, modems, and any other type of device configured to enable data communication via a communication network.

The computing device 200 may further include the one or more sensors 270. In one embodiment, the one or more sensors 270 may include one or more of an image sensor 280, and/or other sensors 290 such as an accelerometer, an optical sensor, an acoustical sensor, an ambient light sensor, a pressure sensor, a contact sensor, an electromagnet sensor, an ECG electrode, an EEG electrode, an EOG electrode, an EMG electrode, and/or a bio impedance sensor, etc. It should be noted that these sensors are only illustrative of a few possibilities and the one or more sensors 270 may include alternative or additional sensors suitable for use in the device 100. It should also be noted that although one or more sensors are described collectively as the one or more sensors 270, any one or more sensors or sensor units within the device 100 may operate independently of any one or more other sensors. Moreover, in addition to collecting, transmitting, and receiving signals or information to and from the one or more sensors 270 at the processor 205, any of the one or more sensor units of the one or more sensors 270 may be configured to collect, transmit, or receive signals or information to and from other components or modules of the computing device 200, including but not limited to the database 240, the I/O module 250, or the interface 260.

As described above with respect to FIG. 1, the accelerometer can be used to detect large-scale motions of a subject indicative of physical activity (e.g., steps, running, walking swimming, etc.) The same accelerometer can be used to determine the onset of a sleep period through the detection of a lack of motion. The acoustical sensor can be used to detect and monitor heart rate. However, in case the sensitivity of the acoustical sensor that detects heart rate is not enough to detect relatively slow heart rate during sleeping, in one embodiment, upon determining that the subject is engaged in sleep, the sensitivity of the acoustical sensor can be reconfigured to detect a significantly lower heart rate. Alternatively, one or more acoustical sensors can be dedicated to, and configured for, detecting relatively slow heart rate during sleeping while one or more other acoustical sensors are used to detect regular heart rate during physical activity.

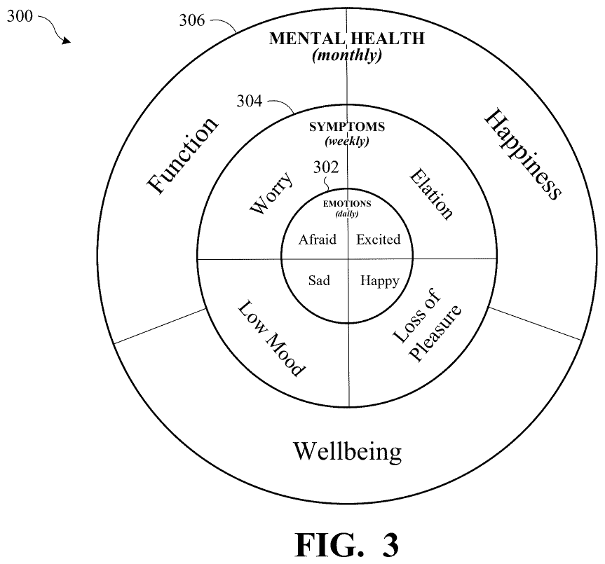

FIG. 3 is an example of a chart 300 that illustrates a mental health assessment in accordance with the present teachings. That is, the chart 300 may illustrate the mental health assessment conducted using the evaluation and guidance system described above and in further detail below. As shown in the chart 300, the mental health assessment may be conducted using a multi-layered approach over different time intervals. For example, the mental health assessment may include a first layer 302, a second layer 304, and a third layer 306. All or a portion of the mental health assessment may be conducted by a portable device of a user (e.g., the device 100 worn by the user).

The first layer 302, the second layer 304, and the third layer 306 may provide a technique to conduct a mental health assessment based upon an emotion-symptom-metric approach. That is, the first layer 302 may correspond to an emotion determination and/or evaluation, the second layer 304 may correspond to a symptom determination and/or evaluation, and the third layer 306 may correspond to a metric (e.g., a mental health metric) determination and/or evaluation. Based upon such layering, the mental health assessment may initially determine one or more emotions of the user in the first layer 302 (i.e., a first stage), whereby the one or more determined emotions of the user may be further evaluated in the second layer 304 (e.g., a second stage) to determine one or more symptoms of the user. Additionally, the one or more determined symptoms and/or the one or more determined emotions may also be further evaluated in the third layer 306 (e.g., a third stage) to determine one or more metrics (e.g., mental health metrics). Thus, the mental health assessment based upon the aforementioned layered approach may begin with initial emotion detection and thereafter determine macro-level parameters (e.g., symptoms and/or mental health metrics) associated with the user.

The first layer 302 may correspond to a daily assessment of an individual (e.g., the user of the device 100) with respect to their mental health. The first layer 302 may correspond to a momentary assessment (e.g., a momentary point in time) of the user. Additionally, or alternatively, the first layer 302 may correspond to an event-related assessment. For example, the event-related assessment may be triggered based upon one or more events (e.g., one or more physiological conditions or triggers).

The first layer 302 of the mental health assessment may be based upon the one or more physiological conditions detected by the portable device (e.g., the device 100). Such physiological conditions may then be evaluated or otherwise utilized to determine one or more associated emotions of the user. By way of example, the device 100 may be worn by the user and may detect an increased heart rate and/or increased blood pressure. Based on such detection, the device 100 or the evaluation system using the device 100 (e.g., the processor 205) may determine that the increased heart rate and/or increased blood pressure may correspond to the user feeling afraid or excited. Such determined emotions may then be tracked (e.g., logged in the database 240).

The determined emotions may be cumulatively tracked on a momentary or event-related basis as well as a daily basis in the first layer 302 such that any determined emotions within a given day are logged. However, it should be noted that an interval of detection may dictate the total number of emotions determined in a given day. By way of example, the device 100 may be configured to detect the one or more physiological conditions every 5 minutes (i.e., based on a 5-minute interval) such that one or more emotions may be determined every 5 minutes and thereby cumulatively tracked throughout the day. The device 100 may also be configured to detect the one or more physiological conditions (e.g., every 5 minutes), and based upon congruent detections (e.g., if sequential emotions determined by sequential physiological conditions detected are the same), the device 100 may transmit a notification to the user for subjective evaluation, as discussed further below. It should be noted that such intervals may vary, such as when the user is sleeping (e.g., less tracking due to inactivity).

As shown in FIG. 3, the first layer 302 may conduct the initial phase (e.g., the first stage) of the mental health assessment based upon determining one or more emotions of the user. The emotions determined based upon the physiological conditions detected may be categorized into four emotion categories, which are represented as four quadrants in the first layer 302. That is, the emotions determined may be tracked based on the emotion categories, which may be represented by fear, sadness, happiness, and excited. For example, the four emotion categories may represent the four quadrants of the arousal and valence characteristics of the particular emotion. Arousal and valence may each be measured as a value for a particular quadrant. For example, an arousal value may be measured as high or low and a valence value may be measured as positive or negative. Thus, based upon the combinations of arousal values and valence values, a particular emotion category (e.g., an emotion category quadrant) may be determined.

The emotions determined based upon the physiological conditions may be categorized and tracked (e.g., logged) as a single one of the above-identified emotion categories or may be categorized and tracked (e.g., logged) as more than one of the above-identified emotion categories. For example, the device 100 may detect one or more physiological conditions (e.g., increased heart rate) of the user and determine that the user is both excited and happy, whereby the cumulative log of emotions for the first layer 302 may track a first emotion category associated with the user being excited and a second emotion category associated with the user being happy.

It should also be noted that an emotion summary may be completed in the first layer 302. That is, an emotion summary for each day or other time interval of the first layer 302 may be provided to the user based on the determinations of the first layer 302. The emotion summary may be provided to the user to illustrate the cumulative emotions tracked through a particular day. For example, the emotion summary may include at least one of: a presence frequency of the one or more detected emotions, a percentage of each of the one or more detected emotions, an intensity of the one or more detected emotions, and an indicator for indication motion change of the user (e.g., physical movement of the user).

Based on the first layer 302, the mental health assessment of the evaluation and guidance system may also include the second layer 304. In the second layer 304, the mental health assessment may evaluate the mental health of the user on a weekly basis or on another desired time interval based upon one or more categorized symptoms. The second layer 304 may compile the emotions (e.g., the emotion categories) tracked in the first layer 302 to extrapolate one or more symptoms of the user. The one or more symptoms of the user may also be evaluated in the second layer 304 based upon severity, frequency, chronicity, or a combination thereof. For example, with respect to mental health, a symptom may be considered for diagnostic purposes if the symptom persists for longer than two weeks and interferes with functioning of the user. Therefore, for the mental health assessment described herein, detection, evaluation, and guidance with respect to symptoms may be conducted on a time interval between one to two weeks.

By way of example, depression, when defined as low mood or a lack of enjoyment (e.g., anhedonia), may be considered a normal state (e.g., a normal emotional state) of the user. Only when the depression persists for more than two weeks and begins impairing function of the user may it become an issue that warrants guidance (e.g., guidance based on the mental health assessment described herein). Thus, the mental health assessment may be configured to bridge the gap between emotional and mental health of the user by creating a reporting schedule (e.g., based on the cumulative track as described above) for the emotions and/or the symptoms of the user.

The second layer 304 may conduct an additional portion of the mental health assessment by evaluating the cumulatively tracked emotions of the user that were tracked over the course of one or more days. For example, as shown in FIG. 3, the second layer 304 may determine one or more symptoms of the user on a weekly basis. That is, the second layer 304 may evaluate the emotions of the user which are tracked over the course of several days to determine the one or more symptoms. As such, the symptoms may correlate with certain emotions that are present for a prolonged period of time.

By way of example, the second layer 304 accumulate the emotion categories detected in the first layer 302 for further evaluation. The second layer 304 may assess weekly emotions according to the results of the detection within the first layer 302. For example, the second layer 304 may create weekly scores for each emotion category detected according to the results of detection within the first layer 302 if the results of detection within the first layer 302 may be categorized as: decreased joy, increased fear, increased excitement, or increased sadness.

That is, for each day, if the emotion accumulation (e.g., the cumulative tracking of the emotion categories determined in the first layer 302) meets the aforementioned conditions (e.g., decreased joy, increased fear, increased excitement, or increased sadness), the second layer 304 may report a value of an effect size (e.g., a severity) of the emotion accumulation. The value of the effect size may be based upon one or more value ranges, which may correspond to the following severity categories: mild, moderate, and severe. While such severity categories are not particularly limited to any range of values, it is envisioned that the mild severity value range may be a range less than the moderate severity value range, and the moderate severity value range may be less than the severe severity value range.

Based on the above, weekly scores associated with determined emotions may fall into one of the above severity categories (e.g., mild, moderate, or severe). Based on the severity, the mental health assessment may provide correlated recommendations, which may be cumulative. For example, if the weekly score associated with the determined emotion is moderate, the mental health assessment may provide recommendations and/or guidance for both mild and moderate protocols associated with the determined emotion.

The symptoms of the second layer 304 may be categorized into four main categories: worry, elation, low mood, and loss of pleasure. These four symptoms may correlate to a prolonged feeling of the user being afraid, excited, sad, and experiencing a lack of enjoyment, respectively. In other words, if the user feels sad frequently over the course of an entire week—that is, sadness is one of the determined emotions that is tracked (e.g., logged) over several days and is significantly present in the tracked emotions data—the second layer 304 of the mental health assessment may determined that the user is experiencing a low mood as a symptom. Such an evaluation may be conducted at the second layer 304 based upon one or more evaluation metrics (e.g., parameters) and is not particularly limited to any one process. For example, as discussed above, the evaluation conducted at the second layer 304 may include one or more thresholds to determine if a particular emotion category represented by fear, sadness, happiness, or excitement is “significantly” present over the course of the week, whereby the evaluation at the second layer 304 may also determine a severity of the emotion category. It should also be noted that, similar to the first layer 302, a subjective cross-validation may be completed in the second layer 304 to confirm the symptoms determined in the second layer 304. For example, the user may be prompted (e.g., via the device 100) to confirm the symptom determined in the second layer 304 to cross-validation such determination.

In addition to evaluating the user's mental health on a momentary, event-related, and/or daily basis in the first layer 302 and a weekly basis in the second layer 304, the mental health assessment may also include a more macro-level assessment of the user's mental health in the third layer 306. In the third layer 306, the mental health of the user may be assessed on a monthly basis or based upon a time interval greater than weekly. That is, the mental health assessment in the third layer 306 may evaluate the overall mental health of the user based upon the emotions determined and tracked on a daily basis over the course of more than one week. For example, the mental health assessment in the third layer 306 may evaluate the overall mental health of the user based upon emotions determined and tracked over an entire month. As such, a sample size of data (e.g., the emotions tracked) may be significantly larger when compared to the evaluations conducted at the first layer 302 and the second layer 304 such that a more macro-level assessment of the user's mental health may be conducted. Additionally, it should be noted that the evaluation in the third layer 306 may also utilize the symptoms determined in the second layer 304. That is, both the determined emotions of the user and the determined symptoms of the user may be utilized to complete the portion of the mental health assessment conducted in the third layer 306.

The mental health of the user determined in the third layer 306, which takes into account the symptoms and emotions of the user determined in the second layer 304 and the first layer 302, respectively, include one or more aspects (e.g., categories). By way of example and as shown in the chart 300, the mental health of the user may be assessed in the third layer 306 in accordance with three categories: function, happiness, and wellbeing. In other words, the portion of the mental health assessment completed in the third layer 306 may evaluate the overall mental health of the user to determine whether the user is able to properly function, whether the user is happy, and/or whether the wellbeing of the user is positive.

The mental health of the user may be assessed in the third layer 306 by completing a determination for each of the three categories above (e.g., function, happiness, and wellbeing). Such a determination may be a measure of the user's mental health based on assessment of the user over months or even years. For example, based upon the first layer 302 and the second layer 304, the third layer 306 may utilize cumulative emotion (e.g., emotion category) and symptom data over a month or months to provide a clear indication as a user's mental health state with respect to the aforementioned categories (e.g., function, happiness, and wellbeing). Additionally, it should be noted that cross-validation (e.g., subjective feedback) may also be provided by the user in the third layer 306 to confirm one or more determinations with respect to the aforementioned categories (e.g., function, happiness, and wellbeing). For example, the user may be prompted (e.g., via the device 100) on a monthly basis to confirm the assessment conducted in the third layer 306.

A composite score for the user's mental health (e.g., wellbeing) may be determined at the third layer 306 based upon the aforementioned categories (e.g., function, happiness, and wellbeing). The composite score may be based on the severity, frequency, and/or chronicity of the symptoms determined in the second layer 304. Additionally, the composite score may also be based upon self-reported measures of happiness, wellbeing, and function (e.g., input by the user, which may be or may include cross-validation as described above). Moreover, the composite score may be based upon or take into account an evaluation of the user's sleep, exercise, and other lifestyle metrics such that the composite score may be associated with the overall wellbeing of the user. The composite score may then be used to create and provide monthly recommendations and/or guidance to the user such that the user may take action to improve their mental health scores (e.g., the composite score of the third layer 306 and/or the symptom severity of the second layer 304).

Similar to the evaluation completed in the second layer 304, the evaluation completed in the third layer 306 to determine the composite score may be based on the cumulative emotions (e.g., the emotion categories) determined in the first layer 302 and the cumulative symptoms determined in the second layer 304. Such emotion categories and symptoms may be cumulatively tracked over a duration of time, such as a month, at which point the evaluation in the third layer 306 may calculate one or more metrics (e.g., metrics for each of wellbeing, happiness, and function), whereby the metrics may also be cross-validated by the user.

For example, the wellbeing of the user may be based upon the presence of one or more of the symptoms determined in the second layer 304 and the severity and/or frequency of such symptoms. A value may be assigned to the one or more symptoms based upon their severity and/or frequency in a similar manner to the severity range values discussed above with respect to the second layer 304. For example, a value may be assigned to the wellbeing of the user, which may fall into one of the following categories: well, mild, moderate, severe. Thus, the value may correlate to a level or status of the user's wellbeing.

Additionally, the happiness of the user may be correlated to a number of days at which enjoyment and excitement of the user are considered to be at a normal level or slightly higher. For example, a value may be assigned to the happiness of the user based upon a total number of days per month when enjoyment and excitement accumulation is not significantly different from normal (e.g., a normal baseline established by historic data or user input) or with a small effect size in a positive direction. The value of the happiness of the user may then fall into one of the following categories: happy, neutral, or unhappy. Thus, the value may correlate to a level or status of the user's happiness.

Moreover, the function of the user may be correlated to the number of days at which fear and sadness of the user are considered to be at a normal level or slightly lower. For example, a value may be assigned to the functionality of the user based upon a total number of days per month when fear and sadness accumulation is not significantly different than normal (e.g., a normal baseline established by historic data or user input) or with a small effect size in the negative direction. The value of the functionality of the user may then fall into one of the following categories: fully functional, neutral, or dysfunctional.

The mental health assessment in the third layer 306 may then incorporate recommendations similar to the recommendations discussed above in the second layer 304 with respect to determined symptoms, but will focus on long-term change over a month or more. Over the course of months, the mental health assessment may also use self-report questions (e.g., input) from the user to modify or otherwise adjust emotion accumulation severity scores to reflect individualized patterns particular to the user, thereby improving the accuracy of the mental health assessment.

Additionally, as mentioned above and described in further detail below, the mental health assessment may include or be used in conjunction with a cross-validation assessment, which may receive input from the user to confirm or reject any findings from the mental health assessment. That is, the user may provide input to confirm or reject any emotions determined, any symptoms determined, or any overall determinations with respect to the user's overall mental health. For example, the user may provide input or confirmation based upon an emotion determination in the first layer 302, a symptom determination in the second layer 304, and a mental wellbeing (e.g., wellbeing, happiness, and function) determination in the third layer 306. As such, the mental health assessment is based upon both objective (e.g., signal-based) data obtained (e.g., obtained by the device 100) and subjective (e.g., user-reported or cross-validated) data.

It should also be noted that evaluation conducted in the first layer 302, the second layer 304, and the third layer 306 may be done in any manner based upon any metrics or parameters. For example, the symptoms in the second layer 304 and/or the overall mental health assessment in the third layer 306 may by evaluated based upon assigned severity scores or other statistical analyses. Such severity scores of statistical analyses may be based upon or utilize the emotions tracked in the first layer 302. As such, the manner in which the symptoms and overall mental health are determined may be modified or otherwise adjusted for the particular user.

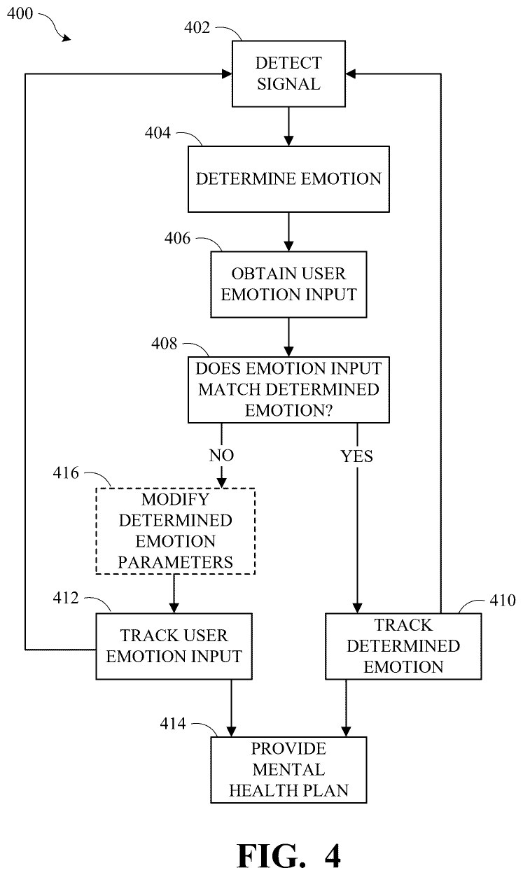

FIG. 4 is a flow diagram illustrating a process 400 for dynamically tracking the emotions of an individual (i.e., a user). The process 400 may be include or be based upon the chart 300 shown in FIG. 3. That is, the process 400 may dynamically track the emotions of the user and evaluate the emotions of the user based upon the chart 300 to complete a mental health assessment.

The process 400 can be implemented as software and/or hardware modules in the computing device 200 of FIG. 2. For example, the process 400 can be implemented as software modules stored in the storage 230 as instructions and/or data executable by the processor 205 of an apparatus, such as the device 100 in FIG. 1. In another example, the process 400 can be implemented in hard as a specialized chip storing instructions executable by the specialized chip. Some or all of the operations of the process 400 can be implemented by the processor 205 of FIG. 2.

As shown in FIG. 4, the process 400 may begin at operation 402 by detecting a signal. The signal detected may be one or more physiological conditions of the user. By way of example, the user may wear the device 100, whereby one or more of the sensors of the device 100 may detect one or more physiological conditions of the user, such heart rate, heart rate variability (HRV), blood pressure, etc. The detected signals at 402 may correspond directly to physiological conditions or may be further evaluated (e.g., by the device 100 and/or the processor 205) to determine the physiological conditions exhibited by the user.

The detected physiological conditions may then be utilized to determine an emotion at 404. That is, the detected physiological conditions may be used to determine one or more emotions felt by the user in that moment in time. In other words, the signal detected at 402 may provide a momentary or isolated detection point, and the emotion category determined at 404 may be the emotion felt by the user for that particular momentary or isolated detection point. By way of example, the signal detection at 402 and the emotion determination at 404 may be part of the first layer 302 of the mental health assessment shown in FIG. 3. For example, the signal may be detected at 402 to determine a physiological condition of the user and an emotion (e.g., afraid, sad, excited, or happy) may be determined at 404 based upon the physiological condition. As discussed above, one or more emotions (i.e., one or more categories of emotion) may be determined at 404 based upon one or more physiological conditions of the user.

Once the emotion is determined at 404, the process 400 may obtain user emotion input at 406. That is, the process 400 may receive input from the user to cross-validate the emotion determined at 404. Based on such validation, the process 400 may objectively collect data (e.g., physiological conditions of the user) to determine an emotion of the user and also subjectively collect data (e.g., user input) to more accurately determine the emotion of the user. The emotion input by the user at 406 may be used to confirm whether the emotion determined at 404 is accurate. That is, the user may confirm at 406 whether the emotion determined at 404 accurately reflects the emotion subjectively felt by the user.

By way of example, the device 100 may be worn by the user and detect a signal that corresponds to a physiological condition of the user, such as an increased heart rate and/or increased blood pressure. Based upon such physiological conditions, the process 400 (e.g., via the device 100, the processor 205, or another device in communication with the device 100, such as a mobile phone of the user) may determine one or more emotions associated with the physiological conditions detected that were experienced by the user. Once the one or more emotions are determined, the user may be prompted, such as using an interface (e.g., a display screen) of the device 100 or an interface of the computing device 200, to confirm whether the one or more emotions determined accurately reflect what the user subjectively experience. As such, the user may input—either confirm or reject—the one or more emotions determined.

After the user emotion input is received at 406, the process 400 may evaluate the emotion input at 408. That is, the process 400 may determine at 408 whether the user confirmed the emotion determined at 404 or rejected the emotion determined at 408. It should be noted that other inputs from the user are possible and may extend beyond confirming or rejecting the determined emotion. For example, the user may provide further feedback at 406 with respect to the emotion determined to better categorize the emotion. The user may input an intensity of the emotion experience and/or determined, one or more physiological conditions felt at that moment, or other feedback, all of which may be input into the process 400 to better evaluate the mental health of the user.

If determined at 408 that the user confirmed the emotion determined at 404, the emotion may be tracked at 410. However, if determined at 408 that the user rejected the emotion determined at 404, the user may input an emotion, which may then be tracked at 412 instead of the emotion determined at 404. That is, the user may input an emotion that is different than the emotion determined at 404, and the emotion input by the user may be tracked instead of the emotion determined at 404 to more accurately log the emotion experienced by the user. By way of example, if the user rejects the emotion determined at 404 (e.g., rejects the emotion as an input at 408), the user may be further prompted (e.g., via an interface of the device 100) to manually input the emotion subjectively experience at that moment, which may then be tracked at 412. As shown in FIG. 4, the process 400 may continuously track both emotions determined based on the physiological conditions detected and cross-validated by the user and emotions manually input by the user.

By way of example, the emotion may be logged and stored as data in the device 100 (e.g., in the storage 230 and/or the database 240) and/or may be stored in an external location, such as a cloud-based storage system. As discussed above with respect to FIG. 3, the emotions may be tracked (e.g., logged) based on any desired schedule or interval (e.g., every 15 minutes, every 30 minutes, every hour, etc.) on a daily basis. Thus, one or more emotions categories as discuss above may be tracked in any given day to create a cumulative total of the emotion categories determined and experienced by the user on a daily basis (e.g., a daily basis accumulation). Therefore, the emotions categories tracked may provide a data set of emotion category data (e.g., the emotion categories represented by fear, sadness, happiness, and excitement), which may be further evaluated to determine the mental health of the user. For example, the emotion category data may be utilized to complete the mental health assembly shown in FIG. 3 in the second layer 304 and/or third layer 306.

Once the emotion either input by the user or determined based upon the physiological conditions detected is tracked at 412 and 410, respectively, a mental health plan may be provided to the user at 414. The mental health plan may be provided to the user in any manner. For example, the mental health plan may be provided to the user via a display or other user interface of the device 100 (e.g., via a speaker), may be provided via an external device, such as a mobile phone or tablet of the user, or a combination thereof.

The mental health plan may be based upon the emotions tracked at 410 and/or 412. For example, the mental health plan provided to the user may be based upon the evaluation or portion of the mental health assembly conducted at the first layer 302, the second layer 304, the third layer 306, or a combination thereof of the chart 300. That is, the mental health plan provided to the user may be based upon one or more of the specific emotions tracked, based upon one or more of the determined symptoms of the user, based upon the overall mental health determination in the third layer 306, or a combination thereof. As such, the mental health plan may be dynamically provided to the user at any point in time at either a micro- or macro-level.

The mental health plan may also include any desired information. The mental health plan may include one or more recommendations for the user. For example, the mental health plan may include one or more recommended activities (e.g., exercises, books, meditations, etc.), one or more recommended actions (e.g., take a shower, eat, rest, etc.), or both. The mental health plan may also recommend when the user may need to speak with a medical professional based upon their current mental health state. For example, if there is a determination based on the emotions tracked at 410 and 412 (e.g., based on the assessment completed at the second layer 304 and/or the third layer 306 of the chart 300) that the user is experiencing ongoing and/or severe emotional distress, the mental health plan provided to the user at 414 may recommend that the user seek medical attention, such as speaking with a therapist.

The mental health plan may also assess the response of the user to any guidance or recommendations provided to the user. Assessment of the response of the user to guidance or recommendations may be assessed as a function of change in symptom severity and/or symptom frequency over one to two weeks subsequently following the guidance or recommendations (e.g., the recommended schedule) similar to the assessment used in psychotherapy. When necessary, the mental health plan may track completion of the guidance or recommendations in response to a detected momentary emotion.

In addition to recommendations, the mental health plan provided to the user may also include various information that may be relevant to the user. For example, the mental health plan may provide medical information with respect to a particular emotion or symptom, may provide contact information for local medical professionals or activities (e.g., a local gym), or may provide information based upon one or more interests of the user (e.g., activities of interest, books from an author of interest, etc.). As such, the mental health plan provided to the user may be tailored to the user based on not only emotions tracked at 410 and/or 412, but also based on various user input.

For example, prior to detecting a signal at 402, the process 400 may require the user to manually complete a questionnaire or answer particular questions to establish a baseline mental health for the user. The questionnaire may also prompt the user to input their interests, dislikes, or other opinions such that the process 400 may be tailored more specifically to their subjective feelings. Therefore, the emotions determined at 404 may also better align with what the user is most likely to experience subjectively.

It should also be noted that the process 400 may dynamically adjust based upon the subjective input (e.g., the cross-validation at 406). For example, one or more emotion parameters may be modified at 416 to adjust how emotions are determined at 404 for subsequently detected physiological conditions.

By way of example, the physiological symptom detected at 402 may be an increased heart rate of the user. Initially, the emotion determined at 404 may be fear, which may be associated with stress and/or anxiety of the user. However, the user may input at 406 that the emotion is incorrect, at which point the user may manually input that they feel excited and happy. Based on such input, the underlying parameters used to determine the emotion at 404 may be adjusted or otherwise modified such that, when that particular physiological condition or combination of physiological conditions (e.g., the increased heart rate) are detected in a subsequent iteration of the process 400, the emotion subsequently determined at 404 in the subsequent iteration may better align with what the user actually experiences-in this case, excitement and/or happiness. Thus, the process 400 may adapt to the user's subjective input to better tailor the process 400 to the user.

It should also be noted that, while an iteration of the process 400 is shown as ended with providing the mental health plan at 414, the process 400 may include one or more ongoing steps beyond 414. For example, as described above, the process may initially provide the user with a self-report questionnaire to establish a baseline for one or more of the emotions and/or symptoms shown in the chart 300. Once such a baseline is established, the process 400 may proceed as described above, resulting in one or more mental health plans being provided to the user at 414. In certain circumstances, the mental health plan provided to the user may provide the user an estimate as to the severity and/or frequency of particular emotions or symptoms.

Additionally, based on the severity and/or frequency, the mental health plan may also provide guidance (e.g., recommendations) as to approaches of psychotherapy that me help alleviate those particular emotions or symptoms. For example, the mental health plan may recommend activities or actions for desensitization if the user exhibits symptoms of stress and/or anxiety (e.g., worry), may recommend cognitive control and behavioral inhibition if the user exhibits symptoms of mania (e.g., elation), or may recommend behavioral activation if the user exhibits symptoms of depression (e.g., low mood).

Based on the above guidance provided in the mental health plan at 414, the process 400 may further monitor or assess the user's response to the guidance. That is, the process 400 may monitor a user's progress with respect to the recommended guidance to determine whether user is actively partaking in the recommended activities. For example, progress of the user in a given activity may be tracked as a function of change in a particular symptom for an interval of time that follows immediately after providing the guidance to the user. By way of example, the process 400 may include one or more additional steps to track a particular emotion or symptom experienced by the user over the course of one to two weeks to evaluate the severity and/or frequency of that symptom. If severity and/or frequency of that symptom has decreased, then it may be determined that the user is successfully partaking in the recommended activities.

However, if severity and/or frequency of the symptom remains unchanged or increases, it may be determined that the user is not partaking in the recommended activities and/or the recommended activities are unsuccessful in helping to alleviate such symptoms, at which point an alternative recommended activity may be provided to the user.

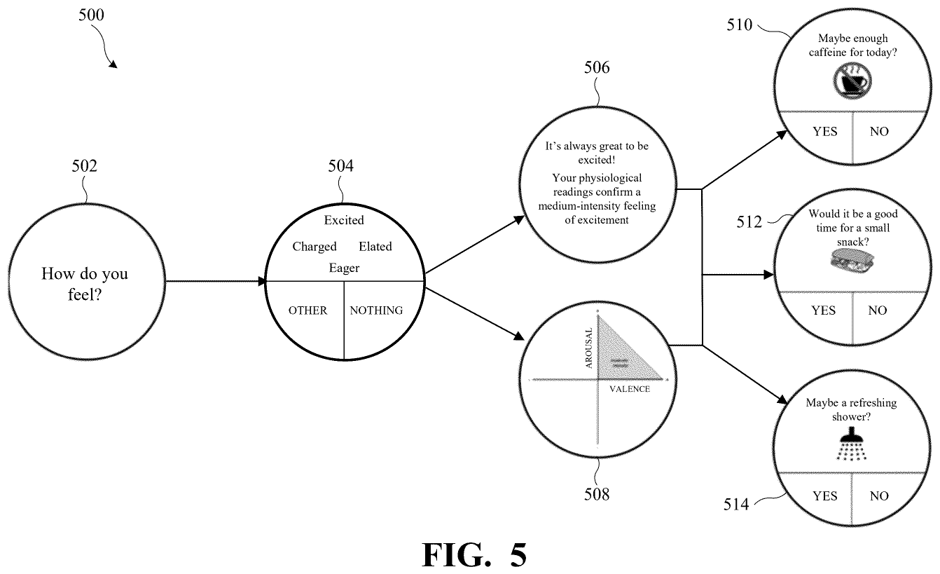

FIG. 5 illustrates a flow diagram 500 of an example of cross-validating emotions with an individual (e.g., with a user) to provide a mental health plan to the user. For example, the flow diagram 500 may be an example of the user interface and/or user input provided in the process 400, such as at operations 406-416. The user interface or screens thereof shown in FIG. 5 may be provided to the user via the device 100 or an external device of the user (e.g., a mobile phone or tablet).

The user interface or screens thereof shown in FIG. 5 can be implemented as software and/or hardware modules in the computing device 200 of FIG. 2. For example, the process shown in the flow diagram 500 can be implemented as software modules stored in the storage 230 as instructions and/or data executable by the processor 205 of an apparatus, such as the device 100 in FIG. 1. In another example, the process shown in the flow diagram 500 can be implemented in hard as a specialized chip storing instructions executable by the specialized chip. Some or all of the operations of the method 700 can be implemented by the processor 205 of FIG. 2.

As shown in FIG. 5, the user may be initially prompted and asked, “How do you feel?” at 502. Such a prompt may occur at, for example, 406 in the process 400 to confirm whether the user is currently feeling the emotion determined at 404. Once the initial prompt at 502 is provided to the user, the user may then see an interface, such as the interface at 504, which may provide the user one or more options to input how the user is subjectively feeling at that moment. For example, as at 504, the user may be prompted to input whether they are feeling excited, charged, elated, eager, nothing, or another feeling. The user may then input their feeling at 504, at which point the user may be provided information via the user interface. For example, the user may input at 504 that they feel excited, and the user interface may provide a new screen at 506 and/or 508, which may provide the user with additional information.

By way of example, 506 and/or 508 may be a mental health plan provided to the user, which may provide the user with information particular to the emotion experienced. Additionally, the mental health plan may provide the user with one or more recommended activities and/or actions, such as those shown in 510-514. That is, once the initial information at 506 and/or 508 is provided to the user, the user may be provided a recommendation to limit their caffeine intake at 510, may be provided a recommendation to eat a small snack at 512, may be provided a recommendation to take a shower at 514, or a combination thereof. The user may input their feedback for one or more of these recommendations such that the evaluation and guidance system described herein (e.g., via the process 400) may record or otherwise utilize their subjective feedback to improve future mental health plans provided to the user. For example, if the user frequently declines take a shower (e.g., 514), subsequent mental health plans provided to the user may no longer suggest taking a shower or may less frequently recommend taking a shower.

Thus, based on the flow diagram 500, it may be gleaned from the present teachings that the evaluation and guidance system described herein may adaptively interact with the user, such as via a user interface of the portable device worn by the user or an external device of the user in communication with the portable device worn by the user, to cross-validate any emotions determined by the system and/or receive other feedback from the user (e.g., feedback pertaining to a particular recommendation).

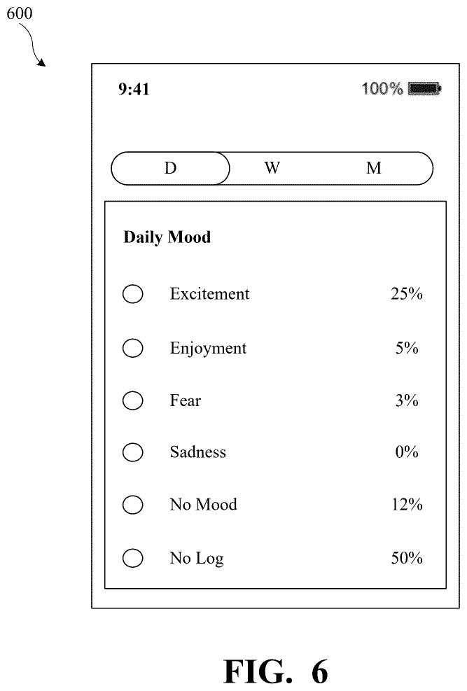

FIG. 6 illustrates an example of a user interface 600 for dynamically tracking the emotions of an individual (e.g. a user). The user interface 600 may be an interface of the portable device worn by the user (e.g., the device 100) or may be an interface of an external device of the user in communication with the portable device worn by the user, such as a mobile phone or tablet of the user. The user interface 600 may be any interface that may facilitate display of information for the user to view. For example, the user interface 600 may be a touch screen or LED display, whereby the user may also provide their input via the user interface 600.

As shown in FIG. 6, the user interface 600 illustrates an example of information provided to the user. As discussed above, the evaluation and guidance system described herein may detect physiological conditions of the user and determine one or more emotions based on those physiological conditions. The determined emotions may then be tracked (e.g., logged) to further evaluate the overall mental health of the user (e.g., to complete a mental health assessment of the user in accordance with the chart 300 shown in FIG. 3).

The emotions may be tracked in a cumulative manner to compile a data set of emotions for evaluation of the overall mental health of the user. For example, the emotions determined may be sorted into the following categories for tracking purposes: excitement, enjoyment, fear sadness, no mood (e.g., no mood detected), no log (e.g., no emotion determined based upon a physiological condition detected). Based upon such tracking, the user may be provided a visual indication via the user interface 600 to illustrate a state of mental health of the user. For example, as shown in FIG. 6, the user interface 600 may provide a breakdown or each emotion category to illustrate how often the user exhibits a particular emotion. Such emotions may be further broken down by day (e.g., a daily mood), by week (e.g., a weekly mood), and by month (e.g., a monthly mood).

It should also be noted that the graphics and illustrations provided to the user are not limited to any one display. For example, the user interface 600 may provide any type of chart, graph, table, text, or other visual display for viewing by the user.

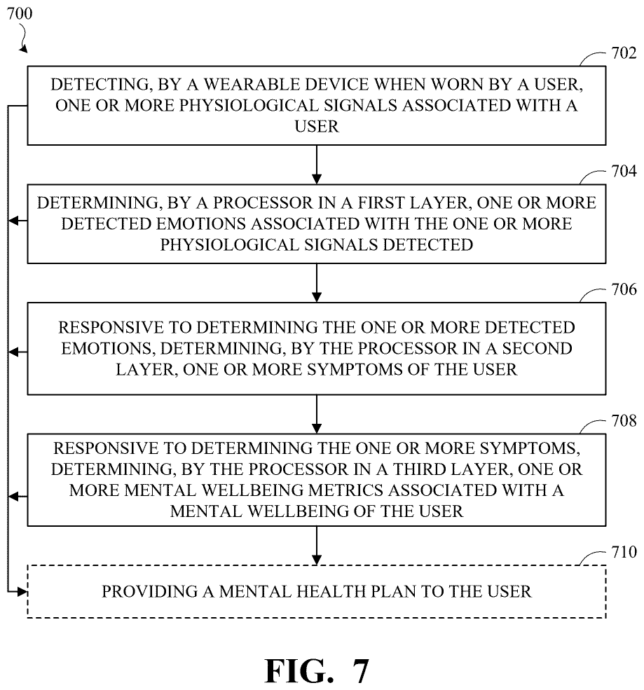

FIG. 7 is a flow diagram of an example of a method 700 for dynamically monitoring emotions of a user using a wearable device. For example, the method shown in FIG. 7 may be implemented using the device 100 and/or one or more external devices in communication with the device 100. The method 700 may be similar to, be part of, or include the process 400 and/or the one or more of the user interfaces shown in the flow diagram 500.

The method 700 can be implemented as software and/or hardware modules in the computing device 200 of FIG. 2. For example, the method 700 can be implemented as software modules stored in the storage 230 as instructions and/or data executable by the processor 205 of an apparatus, such as the device 100 in FIG. 1. In another example, the method 700 can be implemented in hard as a specialized chip storing instructions executable by the specialized chip. Some or all of the operations of the method 700 can be implemented by the processor 205 of FIG. 2.

The method 700 includes detecting, by a wearable device (e.g., the device 100) when worn by the user, one or more physiological signals associated with the user at 702. The one or more physiological signals (e.g., one or more health parameters) may be or may include one or more physiological conditions detected by the wearable device, such as the physiological conditions discussed above.

The method 700 also includes determining, by a processor (e.g., the processor 205 of the computing device 200, which may be in communication with or integrated into the wearable device) in a first layer (e.g., the first layer 302), one or more detected emotions associated with the one or more physiological signals detected at 704. For example, at 704, the physiological conditions detected at 702 may be evaluated to determine one or more emotions of the user. Additionally, the one or more detected emotions may be cumulatively tracked to create a log of the one or more detected emotions.

Responsive to determining the one or more detected emotions at 704, the method 700 may also include determining, by the processor in a second layer (e.g., the first layer 302), one or more symptoms of the user. As discussed above, the one or more symptoms of the user may be based on the one or more detected emotions determined at 704.

Responsive to determining the one or more symptoms, the method 700 may also include determining, by the processor in a third layer (e.g., the third layer 306), one or more mental wellbeing metrics associated with the user. As discussed above, the one or more mental wellbeing metrics may be based on the one or more detected emotions determined at 704 and the one or more symptoms determined at 706.

The method 700 may also include providing a mental health plan to the user. As described above, the mental health plan may be provided to the user and may include one or more recommendations, activities, actions, or other information. The mental health plan may be based on the one or more detected emotions, the one or more symptoms, the one or more mental wellbeing metrics, or a combination thereof.

As described above, a person skilled in the art will note that all or a portion of the aspects of the disclosure described herein can be implemented using a general-purpose computer/processor with a computer program that, when executed, carries out any of the respective techniques, algorithms, and/or instructions described herein. In addition, or alternatively, for example, a special-purpose computer/processor, which can contain specialized hardware for carrying out any of the techniques, algorithms, or instructions described herein, can be utilized.

Similarly, all or a portion of the aspects of the disclosure described herein can be implemented by the device 100 (e.g., by the processor 205 when the computing device 200 is incorporated into the device 100), by a server in communication with the device 100 and/or the computing device 200, or both. Additionally, all or a portion of the aspects of the disclosure described herein (e.g., steps, procedures, processes, etc.) may be performed by the device 100 or a secondary companion device (e.g., a mobile terminal, a client device, other remote device, etc.). For example, a portion of the steps or procedures described herein may be performed by the aforementioned server while another portion of the steps or procedures may be performed by the secondary companion device.

Technical specialists skilled in the art should understand that the implementations in this disclosure may be implemented as methods, systems, or computer program products. Therefore, this disclosure may be implemented in forms of a complete hardware implementation, a complete software implementation, and a combination of software and hardware implementation. Further, this disclosure may be embodied as a form of one or more computer program products which are embodied as computer executable program codes in computer writable storage media (including but not limited to disk storage and optical storage).

This disclosure is described in accordance with the methods, devices (systems), and flowcharts and/or block diagrams of computer program products of the implementations, which should be comprehended as each flow and/or block of the flowcharts and/or block diagrams implemented by computer program instructions, and the combinations of flows and/or blocks in the flowcharts and/or block diagrams. The computer program instructions therein may be provided to generic computers, special-purpose computers, embedded computers or other processors of programmable data processing devices to produce a machine, wherein the instructions executed by the computers or the other processors of programmable data processing devices produce an apparatus for implementing the functions designated by one or more flows in the flowcharts and/or one or more blocks in the block diagrams.

The computer program instructions may be also stored in a computer readable storage which is able to boot a computer or other programmable data processing device to a specific work mode, wherein the instructions stored in the computer readable storage produce a manufactured product containing the instruction devices which implements the functions designated by one or more flows in the flowcharts and/or one or more blocks in the block diagrams.

The computer program instructions may also be loaded to a computer or another programmable data processing device to execute a series of operating procedures in the computer or the other programmable data processing device to produce a process implemented by the computer, whereby the computer program instructions executed in the computer or the other programmable data processing device provide the operating procedures for the functions designated by one or more flows in the flowcharts and/or one or more blocks in the block diagrams.

Apparently, the technical specialists skilled in the art may perform any variation and/or modification to this disclosure by the principles and within the scope of this disclosure. Therefore, if the variations and modifications herein are within the scope of the claims and other equivalent techniques herein, this disclosure intends to include the variations and modifications thereof.

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising”, and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. The terms “at least one of A or B,” “at least one of A and B,” “one or more of A or B,” “A and/or B” used herein mean “A”, or “B” or “A and B”.

While the disclosure has been described in connection with certain embodiments or implementations, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of dynamically monitoring emotions of a user using a wearable device, comprising:
detecting, by the wearable device when worn by the user, one or more physiological signals associated with a user;
determining, by a processor in a first layer of the method, one or more detected emotions associated with the one or more physiological signals detected;
responsive to determining the one or more detected emotions, determining, by the processor in a second layer of the method, one or more symptoms of the user, wherein the one or more symptoms are based on the one or more detected emotions; and
responsive to determining the one or more symptoms, determining, by the processor in a third layer of the method, one or more mental wellbeing metrics associated with a mental wellbeing of the user, wherein the one or more mental wellbeing metrics are based on the one or more detected emotions and the one or more symptoms.

2. The method of claim 1, further comprising:
responsive to determining the one or more detected emotions, prompting the user to confirm or reject the one or more detected emotions, wherein the one or more detected emotions are cumulatively tracked to create a log when the user confirms the one or more detected emotions.

3. The method of claim 2, further comprising:
responsive to the user rejecting the one or more detected emotions, modifying one or more parameters used by the processor to determine the one or more detected emotions.

4. The method of claim 2, wherein if the user rejects the one or more detected emotions, the user is prompted to input one or more input emotions, wherein the one or more input emotions are different than the one or more detected emotions.

5. The method of claim 1, further comprising:
responsive to determining the one or more symptoms, prompting the user to confirm or reject the one or more symptoms; and
responsive to determining the one or more mental wellbeing metrics, prompting the user to confirm or reject the one or more wellbeing metrics.

6. The method of claim 1, further comprising:
providing a mental health plan to the user based upon at least one of the one or more detected emotions, the one or more symptoms, and the one or more mental wellbeing metrics, wherein the mental health plan includes at least one of recommendations, activities, actions, and mental health information.

7. The method of claim 1, wherein the method further comprises:
cumulatively tracking, by the processor in the first layer, the one or more detected emotions within a first time interval to obtain a first emotion summary and within a second time interval to obtain a second emotion summary, wherein the one or more symptoms are determined based on the first emotion summary and the second emotion summary such that the one or more symptoms are based upon a third time interval that combines the first time interval and the second time interval.

8. The method of claim 1, wherein prior to detecting the one or more physiological signals associated with the user, the method further comprises:
receiving, from the user as an input, an emotional baseline that includes one or more expected emotions or a response to a wellbeing question to assess a validity of the one or more detected emotions.

9. The method of claim 1, wherein the one or more detected emotions are categorized based upon an associated arousal signal and an associated valence signal that are derived from the one or more physiological signals, and wherein a combination of the associated arousal signal and the associated valence signal is unique for each of the one or more detected emotions.

10. The method of claim 1, further comprising:
determining, by the processor, at least one of an intensity of the one or more detected emotions and an intensity of the one or more symptoms.

11. A wearable device for dynamically monitoring emotions of a user wearing the wearable device, comprising:
a non-transitory memory; and
a processor configured to execute instructions stored in the non-transitory memory to:
detect, by the wearable device, one or more physiological signals associated with a user;
determine one or more detected emotions associated with the one or more physiological signals detected,
responsive to determining the one or more detected emotions, determine one or more symptoms of the user, wherein the one or more symptoms are based on the one or more detected emotions; and
responsive to determining the one or more symptoms, determine one or more mental wellbeing metrics associated with a mental wellbeing of the user, wherein the one or more mental wellbeing metrics are based on the one or more detected emotions and the one or more symptoms.

12. The wearable device of claim 11, wherein responsive to determining the one or more detected emotions, the processor is further configured to execute the instructions stored in the non-transitory memory to:
prompt the user to confirm or reject the one or more detected emotions, wherein the one or more detected emotions are cumulatively tracked to create a log when the user confirms the one or more detected emotions.

13. The wearable device of claim 12, wherein responsive to the user rejecting the one or more detected emotions, the processor is further configured to execute the instructions stored in the non-transitory memory to:
modify one or more parameters used by the processor to determine the one or more detected emotions.

14. The wearable device of claim 11, wherein a log is created to cumulatively track the one or more detected emotions on a momentary basis, an event-related basis, a daily basis, a weekly basis, and a monthly basis; and
wherein the processor is further configured to execute the instructions stored in the non-transitory memory to:
provide a mental health plan to the user based upon at least one of the one or more detected emotions, the one or more symptoms, and the one or more mental wellbeing metrics, wherein the mental health plan includes at least one of recommendations, activities, actions, and mental health information.

15. The wearable device of claim 14, wherein the one or more detected emotions include one or more emotion categories represented by fear, sadness, happiness, and excitement.

16. The wearable device of claim 11, wherein prior to detecting the one or more physiological signals associated with the user, the processor is further configured to execute the instructions stored in the non-transitory memory to:
receive, from the user as an input, an emotional baseline that includes one or more expected emotions or a response to a wellbeing question to assess a validity of the one or more detected emotions.

17. A non-transitory computer-readable storage medium configured to store computer programs for dynamically monitoring emotions of a user using a wearable device, the computer programs comprising instructions executable by a processor to:
detect, by the wearable device when worn by the user, one or more physiological signals associated with a user;
determine one or more detected emotions associated with the one or more physiological signals detected, wherein the one or more detected emotions are cumulatively tracked to create a log;
responsive to determining the one or more detected emotions, determine one or more symptoms of the user, wherein the one or more symptoms are based on the one or more detected emotions; and
responsive to determining the one or more symptoms, determine one or more mental wellbeing metrics associated with a mental wellbeing of the user, wherein the one or more mental wellbeing metrics are based on the one or more detected emotions and the one or more symptoms.

18. The non-transitory computer-readable storage medium of claim 17, wherein the computer programs further include instructions executable by the processor to:
provide a mental health plan to the user based on at least one of the one or more detected emotions, the one or more symptoms, and the one or more mental wellbeing metrics, wherein the mental health plan includes at least one of recommendations, activities, actions, and mental health information.

19. The non-transitory computer-readable storage medium of claim 17, wherein prior to detecting the one or more physiological signals associated with the user, the computer programs further include instructions executable by the processor to:
receive, from the user as an input, an emotional baseline that includes one or more expected emotions or a response to a wellbeing question to assess a validity of the one or more detected emotions.

20. The non-transitory computer-readable storage medium of claim 17, wherein the one or more detected emotions are categorized based upon an associated arousal signal and an associated valence signal that are derived from the one or more physiological signals, and wherein a combination of the associated arousal signal and the associated valence signal is unique for each of the one or more detected emotions.
